(19) United States
(12) Patent Application Publication
(10) Pub. No.: US 2012/0323166 A1
(43) Pub. Date: Dec. 20, 2012

(54) PHACOEMULSIFICATION IRRIGATION SLEEVE WITH AN ALIGNMENT MARK

(76) Inventors: Matthew J. Fitzgerald, St. Louis, MO (US); Jessica W. Miller, St. Louis, MO (US)

(21) Appl. No.: 13/163,831

(22) Filed: Jun. 20, 2011

(51) Int. Cl. A61F 9/007

(52) U.S. Cl. 604/22

(57) ABSTRACT

A phacoemulsification irrigation sleeve 10 is formed of compliant material. The sleeve 10 is for surrounding at least a portion of a phacoemulsification needle 12 and for transporting irrigation fluid between the needle 12 and the sleeve 10. The sleeve 10 includes at least one irrigation port 16 formed in a shaft 18 of the sleeve 10 near a distal end 20 of the shaft 18. An alignment mark 22 of contrasting color relative to a color of the sleeve 10 is placed on the sleeve 10 in a predetermined orientation with respect to the irrigation port 16.

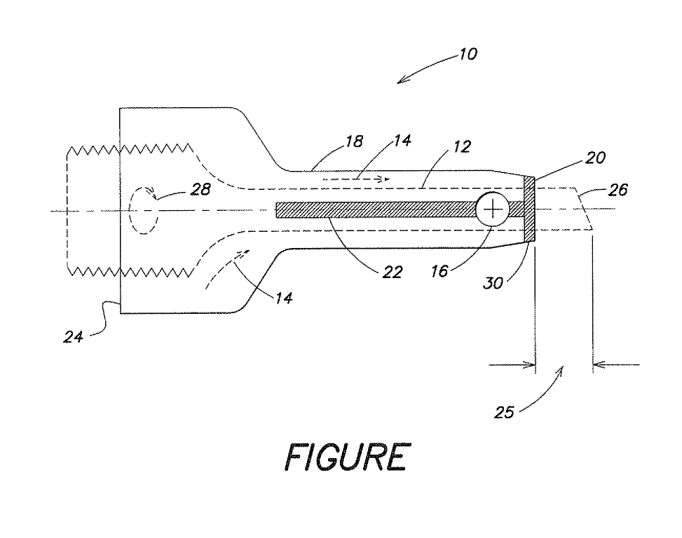

### BACKGROUND

1. Field

The present invention relates to phacoemulsification irrigation sleeves and more particularly, to sleeves with alignment marks relative to irrigation ports formed in the sleeve.

2. Description of Related Art

This section provides background information related to the present disclosure which is not necessarily prior art.

Older prior art irrigation sleeves were opaque and the irrigation ports were easily visible. Newer prior art irrigation sleeves are translucent to enhance the surgeon's view of the surgical site. This translucence benefits the doctor during surgery but presents a challenge to support staff setting-up the instruments prior to surgery. The translucence makes it difficult to see the irrigation ports and align the irrigation ports relative to a bevel of a phacoemulsification needle, in an orientation preferred by the surgeon.

Therefore, a need exists to provide an irrigation sleeve that allows easy alignment of the irrigation ports relative to a phaco needle bevel.

### BRIEF DESCRIPTION OF THE DRAWINGS

The FIGURE is an elevation view of an embodiment of an irrigation sleeve in accordance with the present disclosure.

### DETAILED DESCRIPTION OF CERTAIN PREFERRED EMBODIMENTS

Example embodiments will now be described more fully with reference to the accompanying drawings.

The FIGURE discloses a phacoemulsification (phaco) irrigation sleeve 10. The sleeve 10 is formed of compliant material and is for surrounding at least a portion of a phaco needle 12, shown in dashed-line form. The sleeve 10 is also for transporting irrigation fluid between the needle 12 and the sleeve 10, as represented by dashed arrows 14. The sleeve 10 includes at least one irrigation port 16 formed in a shaft 18 of the sleeve 10 near a distal end 20 of the shaft 18. An alignment mark 22 of contrasting color relative to a color of the sleeve 10 is placed on the sleeve 10 in a predetermined orientation with respect to the irrigation port 16.

The alignment mark 22, in one embodiment, is a stripe positioned along an axis of the shaft 18 extending from or near the distal end 20 towards a proximal end 24 of the sleeve 10, such that the stripe intersects the irrigation port 16, as shown. The alignment mark 22 allows a user to easily and reliably orient the irrigation port 16 relative to the bevel 26 of the phaco needle 12. The alignment mark 22 also allows the user to quickly determine a distance 25 of the sleeve distal end 20 from a distal end of the needle 12.

This contrasting alignment mark 22 is particularly useful with the transparent or generally transparent sleeves used in phaco procedures today. If the sleeve 10 is generally transparent it may be difficult for the set-up staff to see the irrigation ports 16 and orient those ports 16 relative to a needle bevel 26 as required by many surgeons. Having a sleeve 10 with the disclosed alignment mark makes such alignment exceedingly easy. Once the sleeve 10 is placed on the needle 12 a user need only rotate the sleeve 10 relative to the needle 12 (as indicated by arrow 28) until the alignment mark 22 is in the desired orientation. It should be noted that sleeve 10 need not be completely transparent to benefit from the use of alignment mark 22; a transparent sleeve is simply one example. Partially translucent sleeves may also benefit from the use of alignment mark 22.

Alignment mark 22 has been shown as a stripe intersecting irrigation port 16. However, alignment mark 22 may be other than a stripe and may be placed so as not to intersect port 16. Alignment mark 22 enables a user to quickly and easily determine the orientation of the irrigation port 16 relative to the needle bevel 26 and any alignment mark 22 placed on the sleeve 10 enabling such orientation may be considered as part of the present invention. Alignment mark 22 could, for example, be placed 90 degrees relative to the irrigation port 16 and be an arrow, a small line, or a dot, and still enable a user to quickly and easily orient the irrigation port 16 properly.

Alignment mark 22 could also be color-coded so that a particular color is easily mated with a particular phacoemulsification needle. Alignment mark 22 does not have to be on the sleeve. Instead, alignment mark 22 could be in the material, e.g. molded into the sleeve 10, or along the inside of the sleeve 10 and visible to a user. In addition, alignment mark 22 could be a separate piece of metal or other material swaged onto the sleeve 10, or a separate piece of plastic bonded on the sleeve 10. Therefore, as used in this specification, when reference or claim is made to the alignment mark 22 being “on the sleeve”, such other embodiments, as described in this paragraph, should be considered as being incorporated into such expression.

Sleeve 10 may also include a stripe 30, of contrasting color relative to the sleeve 10 color, placed circumferentially adjacent the distal end 20 of the shaft 18.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A phacoemulsification irrigation sleeve comprising:
a sleeve formed of compliant material for surrounding at least a portion of a phacoemulsification needle and for transporting irrigation fluid between the needle and the sleeve;
the sleeve including at least one irrigation port formed in a shaft of the sleeve and near a distal end of the shaft; and
an alignment mark of contrasting color relative to a color of the sleeve placed on the sleeve in a predetermined orientation with respect to the irrigation port.

2. The sleeve of claim 1 wherein the sleeve is generally translucent.

3. The sleeve of claim 1 wherein the alignment mark is color-coded so that a particular color is easily mated with a particular phacoemulsification needle.

4. The sleeve of claim 1 wherein the alignment mark is a stripe positioned along an axis of the shaft and extending from the distal end of the shaft towards a proximal end of the sleeve such that the stripe intersects the irrigation port.

5. The sleeve of claim 1 further including a stripe of contrasting color relative to the sleeve color and placed circumferentially adjacent the distal end of the shaft.
